(19) United States
(12) Patent Application Publication
(10) Pub. No.: US 2024/0342076 A1
(43) Pub. Date: Oct. 17, 2024

(54) ULTRASOUND-RESPONSIVE AQUEOUS TWO-PHASE MICROCAPSULE DESIGN FOR ON-DEMAND PULSATILE DRUG RELEASE

(71) Applicants: The Trustees of Columbia University in the City of New York, New York, NY (US); George Mason University, Fairfax, VA (US)

(72) Inventors: Samuel K. Sia, New York, NY (US); Rachel D. Field, Boston, MA (US); Margaret A. Jakus, New York, NY (US); Parag V. Chitnis, Burke, VA (US)

(21) Appl. No.: 18/618,528

(22) Filed: Mar. 27, 2024

Related U.S. Application Data
(63) Continuation of application No. PCT/US2022/077135, filed on Sep. 28, 2022.
(60) Provisional application No. 63/249,947, filed on Sep. 29, 2021.

(51) Int. Cl. A61K 9/00; A61K 9/50; A61K 38/18; A61K 38/19; A61K 45/06

(52) U.S. Cl. CPC A61K 9/0009; A61K 9/5031; A61K 38/18; A61K 38/19; A61K 45/06

(57) ABSTRACT

An aqueous biphasic microcapsule comprising a core comprising a payload-containing polymer and a shell encapsulating the core, the shell comprising a photopolymerized polymer, wherein the payload-containing polymer and photopolymerized polymer are phase separated, and methods of production and use.

### CROSS-REFERENCE TO RELATED APPLICATIONS

This application is a continuation of PCT International Application No. PCT/US2022/077135, filed Sep. 28, 2022, which claims priority to U.S. Provisional Patent Application No. 63/249,947 filed Sep. 29, 2021, each of which is hereby incorporated by reference in its entirety.

### STATEMENT REGARDING FEDERALLY SPONSORED RESEARCH OR DEVELOPMENT

This invention was made with government support under D20AC00004 awarded by the Defense Advanced Research Projects Agency. The government has certain rights in the invention.

### TECHNICAL FIELD

This disclosure generally relates to the fields of materials science and medicine. More specifically, the disclosure relates to microcapsules for controlled release of small molecule payloads, as well as related compositions and methods.

### BACKGROUND

Localized drug delivery can target intended sites in the body while reducing adverse off-target effects. Many implanted drug-delivery systems, such as nanoparticles, hydrogels, and microdevices, work via passive release or exhibit a pre-programmed drug release profile in contrast to localized drug-delivery systems that are externally triggerable for on-demand release. These remotely-activated systems come in a variety of forms, including DNA-capped nanoparticles, microchips, and microcapsules, and can be responsive to visible and near-infrared (NIR) light, magnetic fields, cell membrane receptors, and enzymes.

Additionally, these systems can target delivery to an entire region of the body or within an individual cell. Triggerable release offers potential advantages of efficacious and selective delivery can be controlled on demand; solutions have included reversible NIR disruption of microcapsule membranes and the controlled erosion of layers of a bulk device to release payload. Ultrasound-triggerable microscale systems such as microbubbles typically require destruction of the drug-delivery vehicle, limiting the triggered effect to a one-time bolus release. Towards personalized medicine, real-time tunable drug release could be important; examples include chrono therapy, which optimizes therapies to account for the body's circadian rhythm, and closed-loop drug delivery that releases a payload in response to defined physiological conditions. Pulsatile delivery systems are largely developed at the macroscale rather than microscale which can be implanted or injected, and stimuli-responsive microdevices for localized and repeated drug delivery remain challenging.

There is a growing interest in using ultrasound in various applications including increasing cell membrane permeability for the direct delivery of pay loads; probing the response of cells to shear stresses; and sorting and transporting cells. Ultrasound is a promising mechanism for actuating release, as it is safe and noninvasive, can penetrate deep into the body, and can be focused to submillimeter dimensions. Ultrasound can cause both heating and cavitation in a material, with either of these effects capable of triggering drug release from an encapsulating material. For heating, disruption of gel structure can release drugs, but demonstrations have been mainly performed with bulk gels, and careful attention must be paid to avoid adverse effects from excessive localized heating.

By comparison, cavitation is the formation, growth, oscillation, and collapse of gas bubbles and begins with the formation or presence of gas pockets or dissolved gasses. Oscillations and collapse of bubbles caused by inertial cavitation can release payload by eroding surfaces, disrupting membranes, and rupturing stiff microparticles. Cavitation has been used in conjunction with microbubbles that are loaded with drugs or injected alongside drug depots to achieve drug delivery, but microbubbles were originally designed for short term diagnostic applications; as such, they have limited lifetimes (less than an hour), are fragile, and can only be triggered only once to achieve a burst release before they are destroyed. Bulk materials have been shown to induce cell death more effectively when they release brief ‘bursts’ of drug rather than release drug continuously at lower dosages. Accordingly, there remains a need for a long-term, controllable drug delivery system capable of precise targeting with minimal toxicity.

### SUMMARY OF THE INVENTION

The present disclosure relates to biphasic microcapsules for controlled release of small molecule pay loads. The biphasic microcapsule comprises a core and a shell, wherein the core comprises dextran and the shell comprises photopolymerized poly(ethylene glycol) diacrylate (PEGDA). In some embodiments, the core is asymmetrically disposed within the shell and the shell does not completely encapsulate the core. In some embodiments, the core comprises an aqueous solution of about 3.75% to about 7.5% dextran having an average molecular weight of about 450 kD to about 650 kD. In some embodiments, the shell comprises an aqueous solution of about 10% to about 20% of PEGDA having an average molecular weight of about 10 kD and about 5% to about 30% of PEGDA having an average molecular weight of 575 D. In some embodiments, the core comprises an aqueous solution of about 3.75% to about 7.5% dextran having an average molecular weight of about 450 kD to about 650 kD and the shell comprises an aqueous solution of about 10% to about 20% of PEGDA having an average molecular weight of about 10 kD and about 5% to about 30% of PEGDA having an average molecular weight of 575 D. In some embodiments, the biphasic microcapsule further comprises a drug or other therapeutic composition.

In some embodiments, the present disclosure relates to methods of manufacturing a biphasic microcapsule, comprising: (a) combining an aqueous solution comprising dextran, poly(ethylene glycol) diacrylate (PEGDA), and a photopolymerizing agent in a microfluidic device so as to form a biphasic microcapsule comprising a core comprising the dextran and a shell comprising the PEGDA, wherein the shell encapsulates the core; and (b) subjecting the biphasic microcapsule to a light source to photopolymerize the PEGDA.

In some embodiments, the microfluidic device comprises separate channels for the dextran and PEGDA. In some embodiments, the microfluidic device comprises a junction containing an oil phase and the aqueous solution comprising the dextran, the poly(ethylene glycol) diacrylate (PEGDA), and the photopolymerizing agent is passed through the oil phase. In some embodiments, the oil phase further comprises a surfactant. In some embodiments, the surfactant comprises sorbitane oleate (SPAN™). In some embodiments, the surfactant is present in the oil phase at about 0.5% to about 10% (w/v).

In some embodiments, the present disclosure relates to a method of delivering a drug or other therapeutic composition to a subject comprising administering to the subject one or more biphasic microcapsules containing a therapeutically effective amount of the drug or therapeutic composition and subsequently triggering release of the drug or therapeutic composition by applying focused ultrasound (FUS) to the subject thereby triggering controlled release of the drug or therapeutic composition from the one or more biphasic microcapsules into the subject. In some embodiments, the focused ultrasound is applied to the subject at a frequency of about 1.1 MHz to about 3.3 MHz. In some embodiments, the

focused ultrasound is applied to the subject for about 5 seconds to about 90 seconds. In some embodiments, the focused ultrasound is applied to the subject multiple times over a short period of time (e.g., minutes or hours).

The foregoing is a summary and thus contains, by necessity, simplifications, generalizations, and omissions of detail; consequently, those skilled in the art will appreciate that the summary is illustrative only and is not intended to be in any way limiting. Other aspects, features, and advantages of the methods, compositions and/or devices and/or other subject matter described herein will become apparent in the teachings set forth herein. The summary is provided to introduce a selection of concepts in a simplified form that are further described below. This summary is not intended to identify key features or essential features of the claimed subject matter, nor is it intended to be used as an aid in determining the scope of the claimed subject matter.

### BRIEF DESCRIPTION OF THE DRAWINGS

FIGS. 1A-1D illustrate the microfluidic and triggered release of model drug from biphasic microcapsules. FIG. 1A is a schematic diagram of the fabrication and UV polymerization of biphasic microcapsules in a micro fluidic device. A mixed high and low MW PEGDA and a high MW dextran are used as the two phases. Figure IB is a schematic diagram of the biphasic microcapsule with a dextran inner phase loaded with FITC-dextran as a model drug (green) and a UV-polymerized mixed-MW PEGDA outer phase that is impermeable to payload (light grey). FIG. 1C is a schematic diagram of focused ultrasound transducer being used to selectively trigger a microcapsule. The focal area of the transducer is small enough to selectively actuate individual microcapsules Figure ID shows focused ultrasound is applied to the microcapsule, the payload is released into the surrounding environment.

FIGS. 2A-2D show the on-chip microfluidic fabrication of microcapsules. FIG. 2A shows microfluidic chip design, with regions of interest shown in boxes (B-D correspond to FIGS. 2B-2D). chip, featuring two flow-focusing junctions followed by a serpentine area that allows for sufficient time to complete phase separation and for ultraviolet exposure (purple) to polymerize the PEGDA phase. FIG. 2B shows brightfield (left) and fluorescent (right) images of the first junction, where the dextran phase loaded with model drug co-flows within the outer PEGDA phase. The dotted box in the fluorescent image indicates where mixing occurs along the boundary between the dextran and PEGDA. FIG. 2C shows a brightfield image of the second junction, demonstrating dripping of the dextran and PEGDA phases into the continuous outer oil phase to form discrete aqueous droplets. FIG. 2D shows a brightfield image of the dextran-PEGDA aqueous droplet, undergoing internal phase separation, in the oil phase. (Scale bars=400 pm.)

FIGS. 3A-3D show bulk phase separation and de-mixing of samples. FIGS. 3A and 3B show how samples were made from a mixture of 7.5% dextran 500 k Da and 20% PEGDA 10 k Da, as shown with the original image (FIG. 3A) and the processed image used for analysis (FIG. 3B). Each of the samples at these concentrations has a visibly apparent line, demonstrating phase separation. A solution of 45 pF 50% PEGDA 575 Da with 55 pL 15% Dextran 500 k Da was mixed together and allowed to phase separate overnight. Before heating, the solution consisted of two phases (FIG. 3C). After heating to 80° C., the solution still consisted of two phases (FIG. 3D).

FIG. 4A shows the brightfield (left) and fluorescent (right) images of microcapsules after UV polymerization and washing. FIG. 4B shows the brightfield (left) and fluorescent (right) images of microcapsules taken at 112 days after fabrication. Scale bars=200 pm. FIG. 4C shows the frequency distribution of the microcapsule diameters, post-polymerization.

FIGS. 5A and 5B shows the effects of polymer concentration on phase separation and swelling for optimized microcapsule design. FIG. 5A: (top) Phase diagram showing phase separation of PEGDA 575 and dextran 500 k at various concentrations. Above a certain concentration of each polymer, the one phase solution separated into a dextran-rich and a PEGDA rich phase, (bottom) Phase diagram showing phase separation of PEGDA 10 k and dextran 500 k at various concentrations. Phase separation occurred at lower concentrations of both polymers than when PEGDA 575 was used. FIG. 5B: Swelling ratio of various PEGDA concentrations and compositions. “Mixed MW” PEGDA hydrogels showed low swelling, closer low MW PEGDA (575 MW) than high MW (10 k), suggesting a small pore size similar to low MW PEGDA that could be useful in the outer phase for containing background release of payload in the inner phase.

FIGS. 6A-6D illustrate variations on microcapsule composition. FIG. 6A: Microcapsules formed using an inner phase of 3.75% Dextran 450-650 kDa+2% Dextran FITC 20 kDa, and outer phase of 20% (w/v) PEGDA 10 k MW+30% (v/v) PEGDA 575 MW, and 0.3% FAP (w/v) as the photoinitiator. FIG. 6B: Microcapsules formed using an inner phase of 3.75% Dextran 450-650 kDa+0.6% Dextran FITC 150 kD, an outer phase of

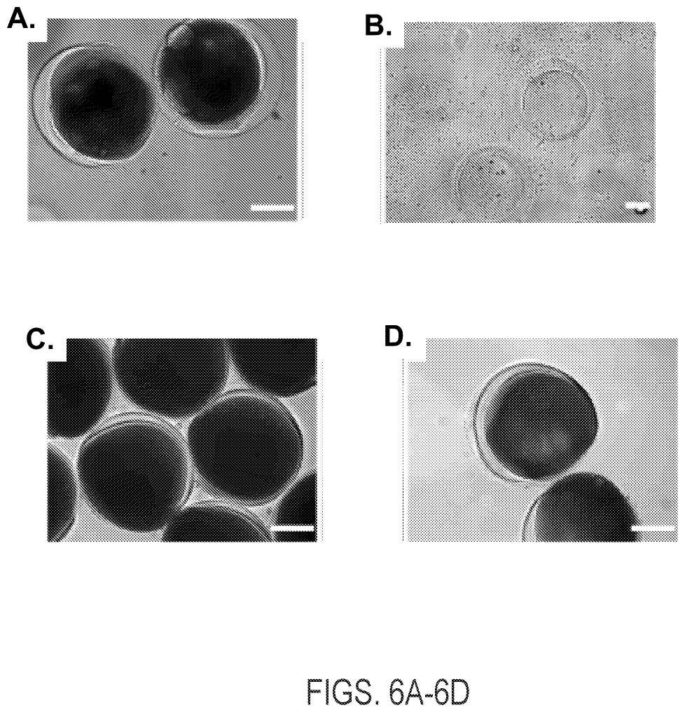

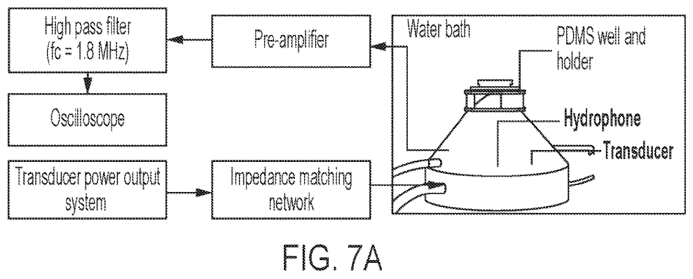

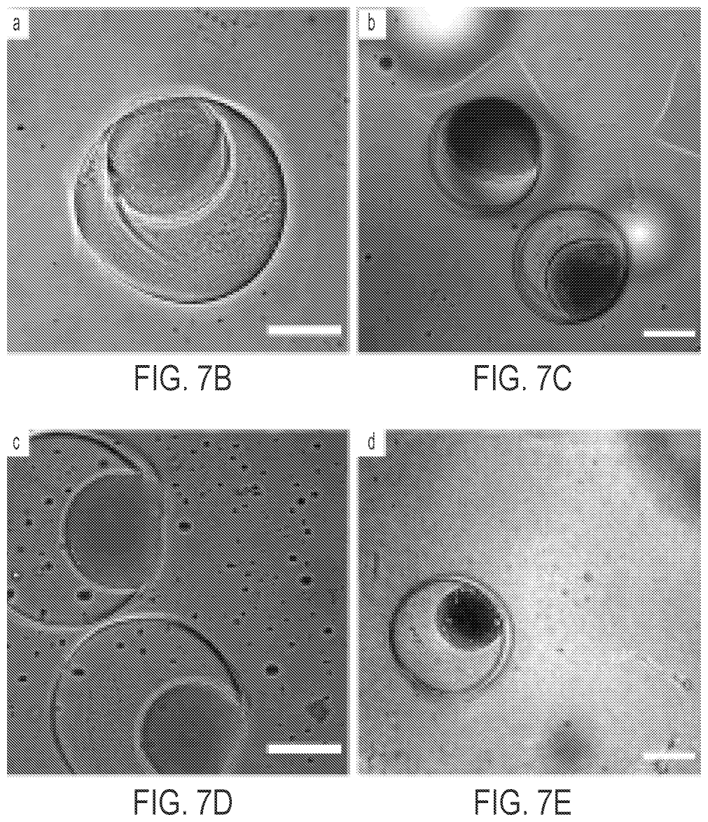

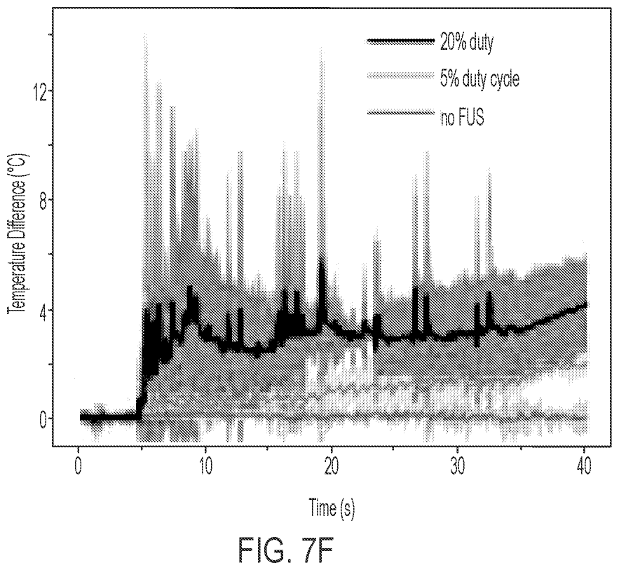

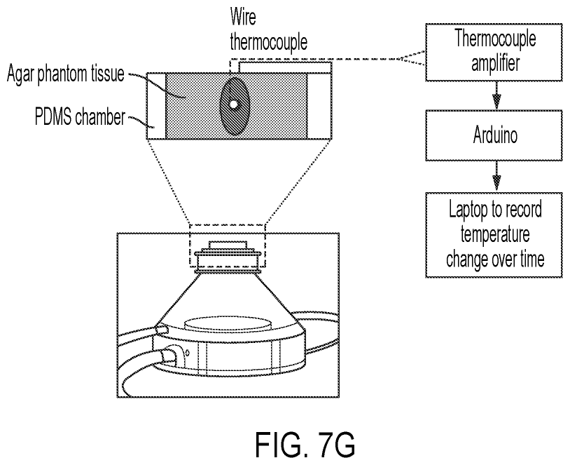

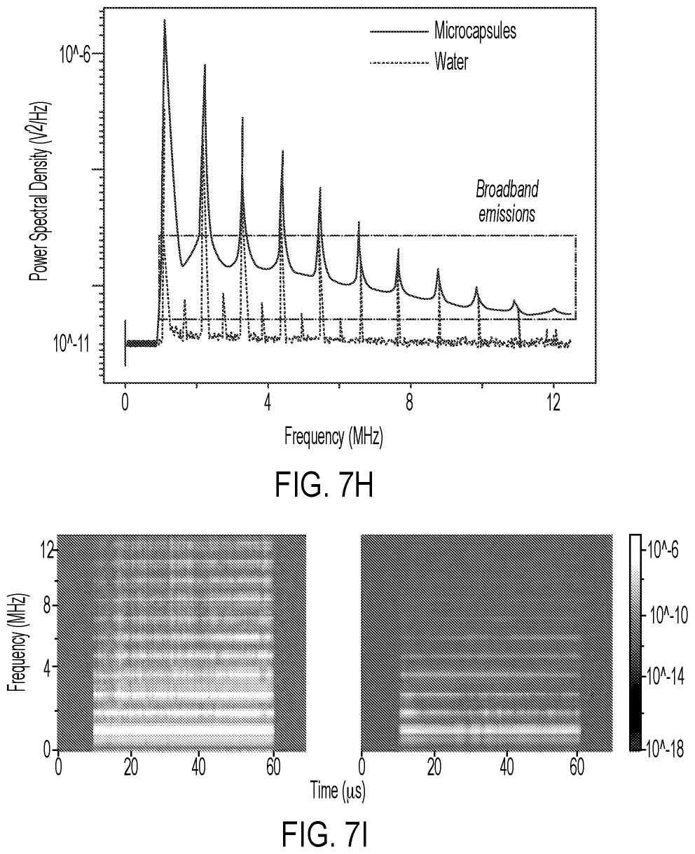

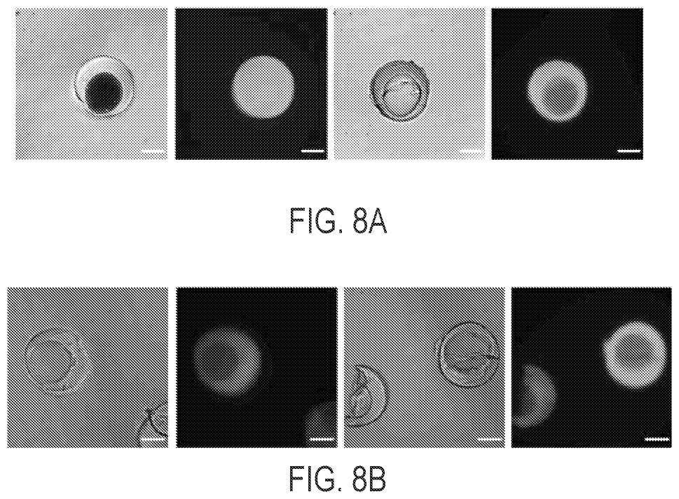

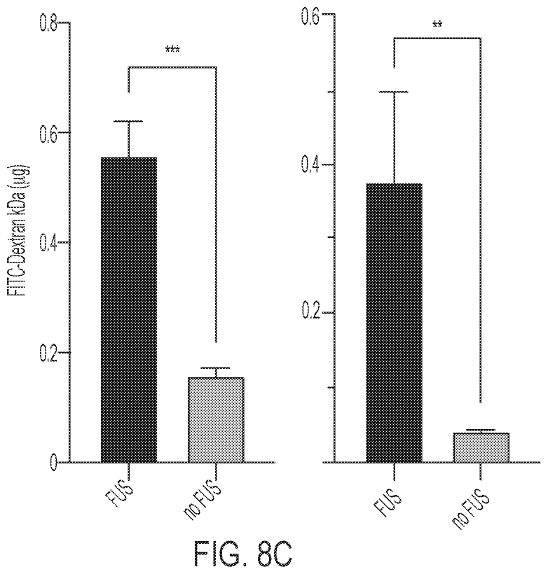

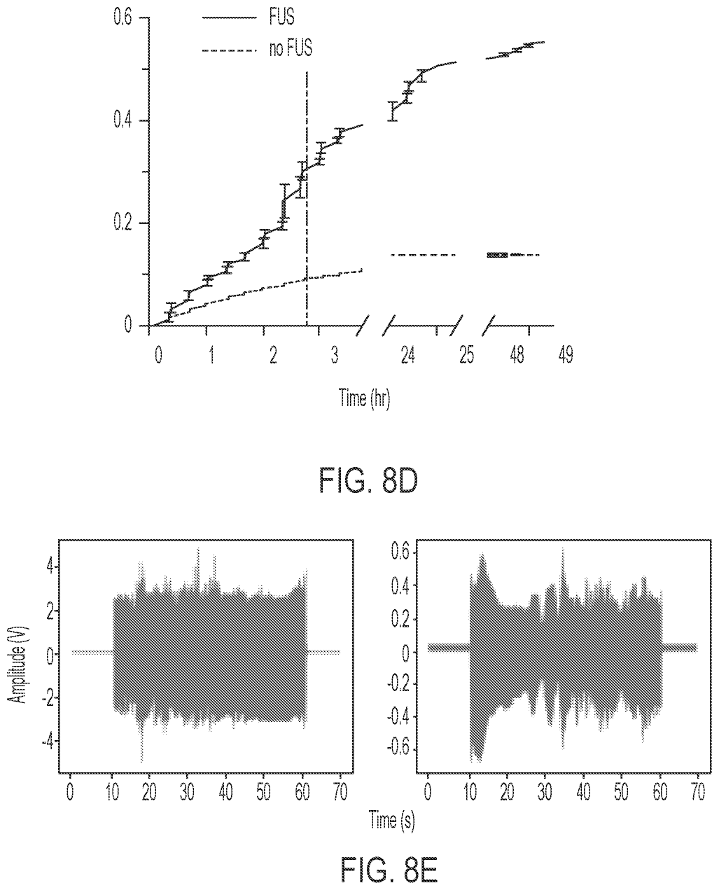

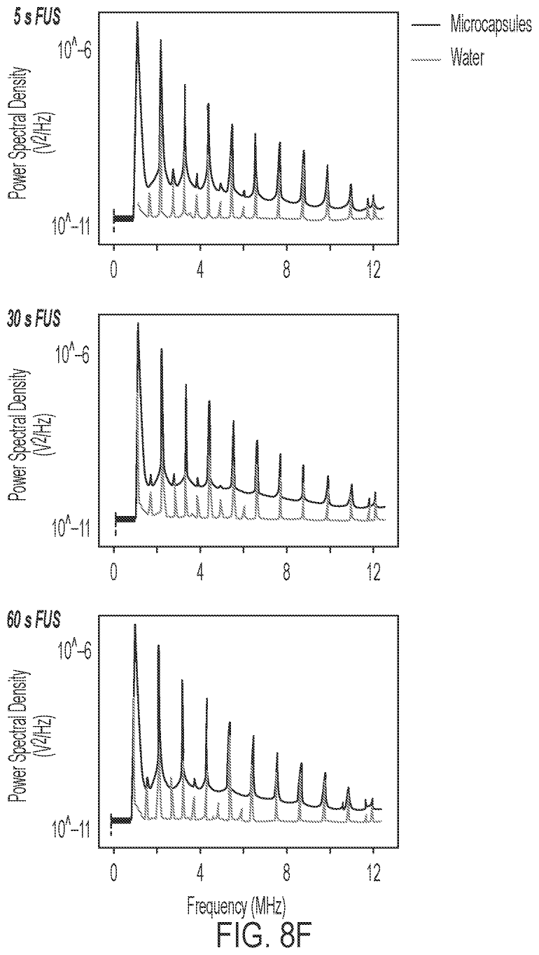

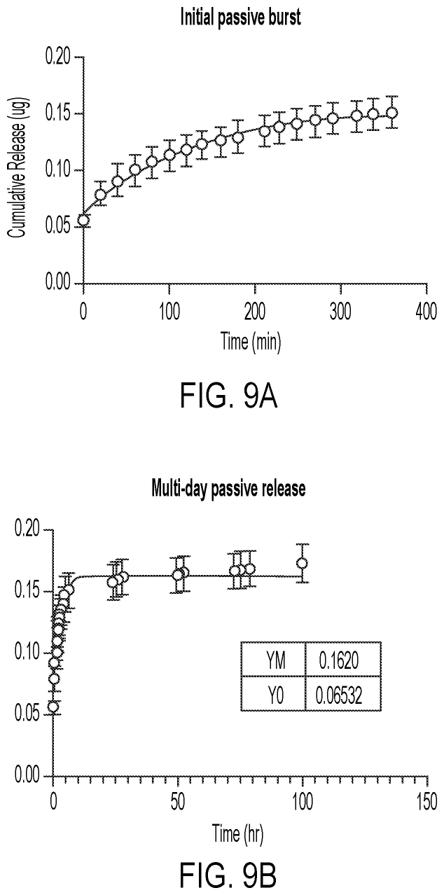

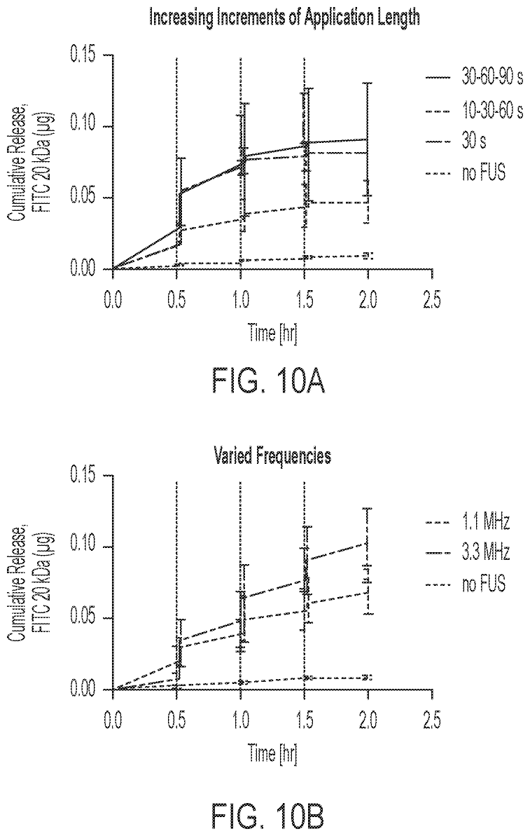

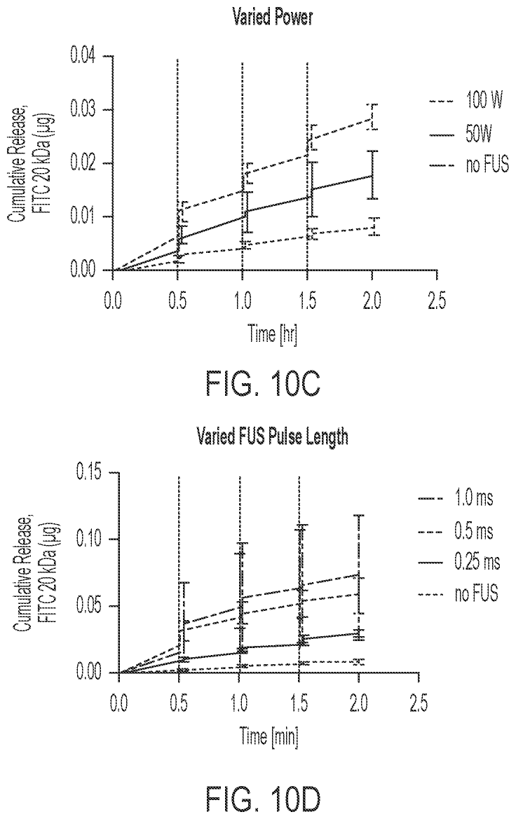

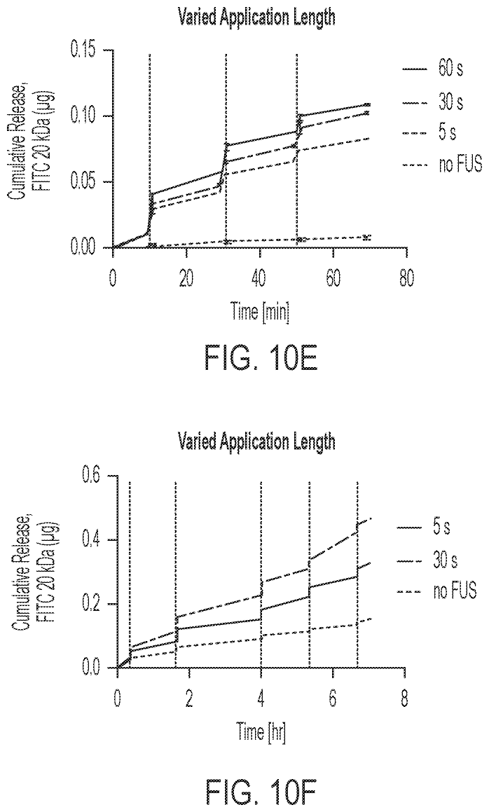

14% (w/v) PEGDA 10 k MW+5% (v/v) PEGDA 400 MW, and 2.5% (w/v) Darocur 2959 as the photoinitiator. In the resulting microcapsules, the inner phase migrates out of the PEGDA structure. FIG. 6C: Microcapsules formed using an inner phase of 3.75% Dextran 500 kDa+2% FITC-Dextran 20 kD, an outer phase of 10% (w/v) PEGDA 10 k MW+30% (v/v) PEGDA 575 MW, and 2.5% (w/v) Darocur 2959 as the photoinitiator. FIG. 6D:
Microcapsules formed using an inner phase of 3.75% Dextran 500 kDa+2% FITC-Dextran 20 kDa, an outer phase of 20% (w/v) PEGDA 10 k MW+30% (v/v) 575 MW, and 2.5% (w/v) Darocur 2959 as the photoinitiator. Microcapsules were formed by premixing dextran and PEGDA and allowed to phase separate prior to being pumped onto the microfluidic chip, rather than in real-time while on-chip. (Scale bars=200 pm).

FIG. 7A-7I show the FUS setup and results, showing inertial cavitation and minimal heating in the microcapsules. FIG. 7A is a block diagram of the FUS experimental setup for FUS application and data processing. FIGS. 7B-7E show the adjustment of the surfactant concentration, to demonstrate modification of the interfacial tension between aqueous and oil phase, with (FIG. 7B) 0.5% Span 80, (FIG. 7C) 2% Span 80. (FIG. 7D) 5% Span 80, and (FIG. 7E) 10% Span 80. (Scale bars=200 pm). FIG. 7F shows the measured thermal effects of 30 s pulsed FUS application, with first FUS application occurring at t=5 s. Shaded areas represent 95% confidence intervals. FIG. 7G shows the thermal responsiveness to FUS application. Experimental test setup for assessing the associated thermal effects from the FUS application. This is a modification of the setup used for cavitation and release profile experimentation, to allow for the placement of a thermistor within immediate proximity to a microcapsule. In the above diagram, the focal area of the transducer is shown within the dotted ellipsoidal area. FIG. 7H shows the power spectral density of the hydrophone signal with microcapsules in the sample well compared to only degassed water in the well. Broadband noise (dotted green box) was observed when the capsules are present, indicative of inertial cavitation. FIG. 7I shows the spectrogram of microcapsule sample (left) and degassed water (right) over a 50 ms pulse of FUS. Increased brightness (yellow) is indicative of increased cavitation.

FIGS. 8A-8D show FUS-triggered release. FIG. 8A: Microcapsule shown prior to FUS in brightfield and fluorescence (left), and after 10 FUS applications at 150 W and 5% duty cycle in brightfield and fluorescence (right). FIG. 8B: Microcapsules after 16 pulsed FUS applications, with the dextran portion of the microcapsule fully released and pitting in the PEGDA phase (left) and cracking of the microcapsule structure (right). FIG. 8C: Total FITC-dextran released over three days (left) and total FITC-dextran released over three days when excluding the initial 2 hours equilibration period, during which surface leaching occurs (right). ***P<0.006, **P<0.0089. FIG. 8D: Release profile of FITC-dextran from a microcapsule undergoing repeated FUS applications over three days, compared to a control sample that does not undergo FUS. FUS was applied for 30 s every 20 minutes, for 10 cycles on day 0 and 3 cycles on days 1 and 2. The dashed line demarcates the end of the equilibration period for the passively releasing microcapsules. Scale bars=200 μm.

FIGS. 8E and 8F show the output of hydrophone-recorded information. FIG. 8E: Recorded output from the hydrophone during a single FUS pulse application, as passed through the amplifier and high-pass filter and recorded via the oscilloscope. FIG. 8F: Power spectral densities of FUS actuations of 5, 30, and 60 s burst lengths.

FIGS. 9A-9B show the passive release from microcapsules. Release profiles for the initial 6 hours (FIG. 9A) and full release of a microcapsule when not exposed to FUS at any point (FIG. 9B).

FIGS. 10A-10F shows the cumulative release of model drug from a microcapsule, as affected by changing a FUS parameter and compared to a negative control sample that does not undergo FUS. Periods of pulsed FUS application shown as yellow bands on the plots. All experimental runs were performed using FUS at 5% duty cycle. The cumulative release corresponding to FUS parameters, as demonstrated by modifying via: (FIG. 10A) stepwise increases of the FUS application period, as 10 s for the first application, 30 s for the second application, and 60 s for the third application, as compared to 30 s for the first application, 60 s for the second application, and 90 s for the third application, and compared to samples which undergo 30 s FUS applications for all three blasts; (FIG. 10B) fₒ frequency, 1.1 MHZ, and f₃ frequency, 3.3 MHz; (FIG. 10C) FUS power intensities; (FIG. 10D) FUS pulse lengths; (FIG. 10E) FUS application times with long periods between FUS applications; and (FIG. 10F) FUS application times with short periods between FUS applications.

### DETAILED DESCRIPTION OF THE PREFERRED EMBODIMENTS

While the present invention may be embodied in many different forms, disclosed herein are specific illustrative embodiments thereof that exemplify the principles of the invention. It should be emphasized that the present invention is not limited to the specific embodiments illustrated. Moreover, any section headings used herein are for organizational purposes only and are not to be construed as limiting the subject matter described.

The embodiments described herein relate to biphasic microcapsules that encapsulate small molecule payloads and permit the controlled release of those payloads using focused ultrasound as described herein.

### Biphasic Microcapsules and Fabrication

The present disclosure relates to biphasic microcapsules for controlled release of small molecule pay loads. The biphasic microcapsule comprises a core and a shell, wherein the core comprises dextran and the shell comprises photopolymerized poly(ethylene glycol) diacrylate (PEGDA). In some embodiments, the core is asymmetrically disposed within the shell and the shell does not completely encapsulate the core. In some embodiments, the core comprises an aqueous solution of about 3.75% to about 7.5% dextran having an average molecular weight of about 450 kD to about 650 kD. In some embodiments, the shell comprises an aqueous solution of about 10% to about 20% of PEGDA having an average molecular weight of about 10 kD and about 5% to about 30% of PEGDA having an average molecular weight of 575 D. In some embodiments, the core comprises an aqueous solution of about 3.75% to about 7.5% dextran having an average molecular weight of about 450 kD to about 650 kD and the shell comprises an aqueous solution of about 10% to about 20% of PEGDA having an average molecular weight of about 10 kD and about 5% to about 30% of PEGDA having an average molecular weight of 575 D. In some embodiments, the biphasic microcapsule further comprises a drug or other therapeutic composition.

In some embodiments, the present disclosure relates to methods of manufacturing a biphasic microcapsule, comprising: (a) combining an aqueous solution comprising dextran, poly(ethylene glycol) diacrylate (PEGDA), and a photopolymerizing agent in a microfluidic device so as to form a biphasic microcapsule comprising a core comprising the dextran and a shell comprising the PEGDA, wherein the shell encapsulates the core; and (b) subjecting the biphasic microcapsule to a light source to photopolymerize the PEGDA.

In some embodiments, the microfluidic device comprises separate channels for the dextran and PEGDA. In some embodiments, the microfluidic device comprises a junction containing an oil phase and the aqueous solution comprising the dextran, the poly(ethylene glycol) diacrylate (PEGDA), and the photopolymerizing agent is passed through the oil phase. In some embodiments, the oil phase further comprises a surfactant. In some embodiments, the surfactant comprises sorbitane oleate (SPAN™). In some embodiments, the surfactant is present in the oil phase at about 0.5% to about 10% (w/v).

### Therapeutic Methods and Compositions

The embodiments described herein also encompass methods of delivering a drug or other therapeutic composition to a subject comprising administering to the subject one or more biphasic microcapsules containing a therapeutically effective amount of the drug or therapeutic composition and subsequently triggering release of the drug or therapeutic composition by applying focused ultrasound (FUS) to the subject and trigger controlled release of the drug or therapeutic composition from the one or more biphasic microcapsules. Anti-cancer agents, such as growth factors and cytokines, as well as pain relievers and insulin are among the drugs contemplated for use with the biphasic microcapsules described herein.

In some embodiments, the focused ultrasound is applied to the subject at a frequency of about 1.1 MHz to about 3.3 MHz. In some embodiments, the focused ultrasound is applied to the subject for about 5 seconds to about 90 seconds. In some embodiments, the focused ultrasound is applied to the subject multiple times over a short period of time (e.g., minutes or hours).

As used herein, a “therapeutically effective” amount refers to the amount of a drug or therapeutic composition that is known (or believed) in the art to treat a particular disease or ailment. As used herein, “treatment” or “treating” or “treat” refers to all processes wherein there may be a slowing, interrupting, arresting, controlling, stopping, alleviating, or ameliorating symptoms or complications of the disease or ailment, but does not necessarily indicate a total elimination of all disease or all symptoms.

In some embodiments, biphasic microcapsules may be delivered to a subject in the form of a pharmaceutical composition, which may comprise one or more pharmaceutically acceptable carriers, diluents, or excipients. Pharmaceutical compositions may be formulated as desired using art recognized techniques. Various pharmaceutically acceptable carriers, which include vehicles, adjuvants, and diluents, are readily available from numerous commercial sources. Moreover, an assortment of pharmaceutically acceptable auxiliary substances, such as pH adjusting and buffering agents, tonicity adjusting agents, stabilizers, wetting agents, and the like, are also available. Certain non-limiting exemplary carriers include saline, buffered saline, dextrose, water, glycerol, ethanol, and combinations thereof. Biphasic microcapsules described herein can be formulated for injection.

Particular dosage regimens, i.e., dose, timing, and repetition, will depend on the particular subject being treated and that subject's medical history. Empirical considerations such as pharmacokinetics will contribute to the determination of the dosage. Frequency of administration may be determined and adjusted over the course of therapy and a therapeutically effective dose may depend on the mass of the subject being treated, his or her physical condition, the extensiveness of the condition to be treated, and the age of the subject being treated.

### EXAMPLES

The following examples have been included to illustrate aspects of the inventions disclosed herein. In light of the present disclosure and the general level of skill in the art, those of skill appreciate that the following examples are intended to be exemplary only and that numerous changes, modifications, and alterations may be employed without departing from the scope of the disclosure.

### Example 1

### Materials

Poly(ethylene glycolj-diacrylate (PEGDA, Polysciences, Mw 10,000), Poly(ethylene glycolj-diacrylate (PEGDA, Sigma-Aldrich, Mw 575), Dextran from Leuconostoc spp. (Sigma-Aldrich, Mw 450,000-650,000) were used in the composition of the droplets. Fluorescein isothiocyanate-dextran (FITC-Dextran, Sigma-Aldrich, Mw 20,000) was used as the model drug. 2-Hydroxy-2-methylpropiophenone, 97% (Sigma-Aldrich) was used as the photoinitiator. Light mineral oil (Sigma-Aldrich) was used for the continuous flow and SPAN® 80 (Sigma-Aldrich) was used as the surfactant.

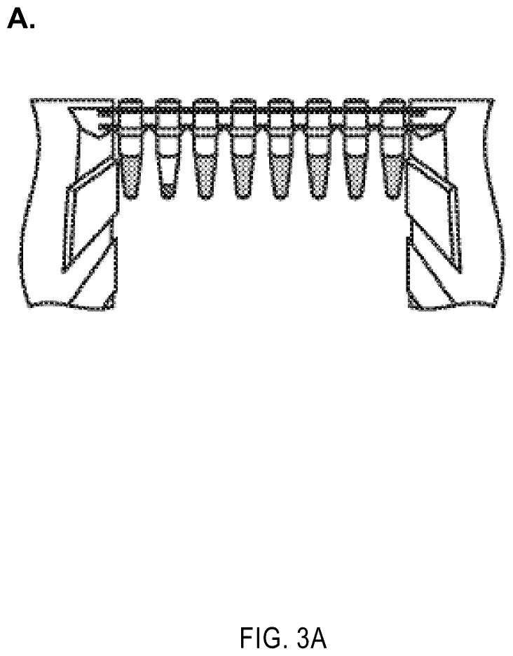

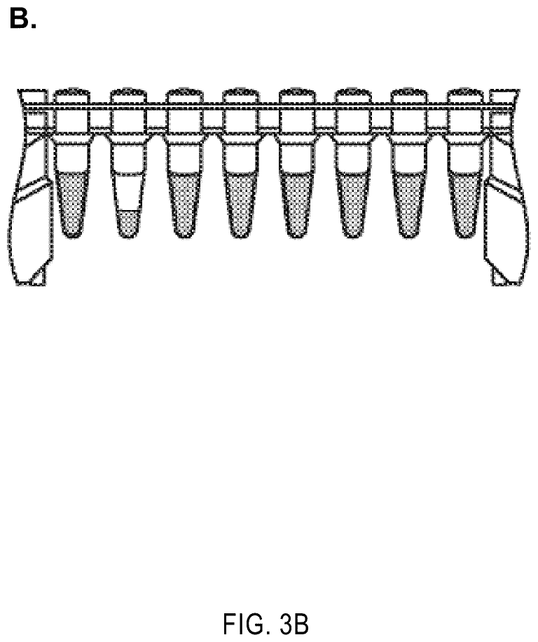

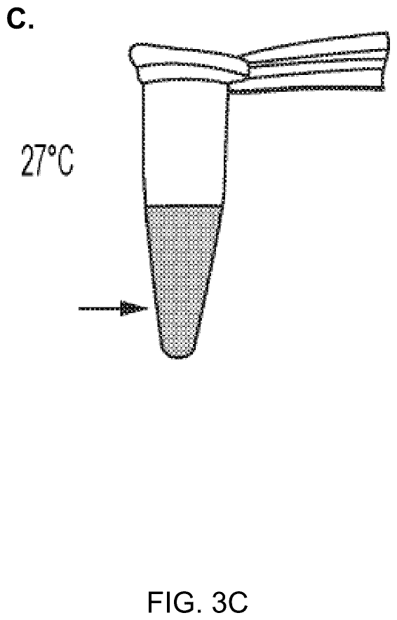

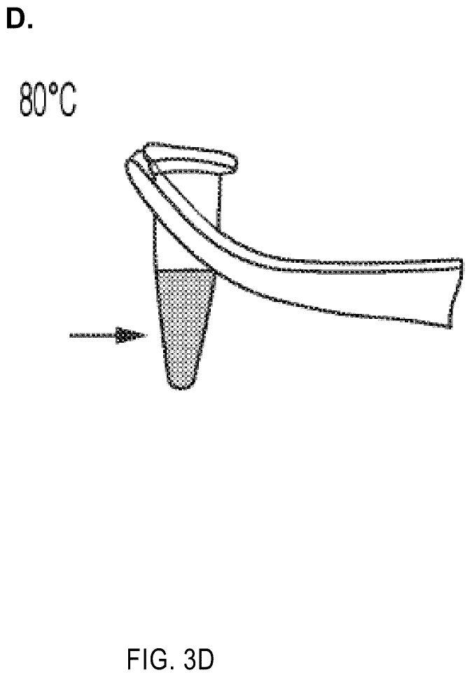

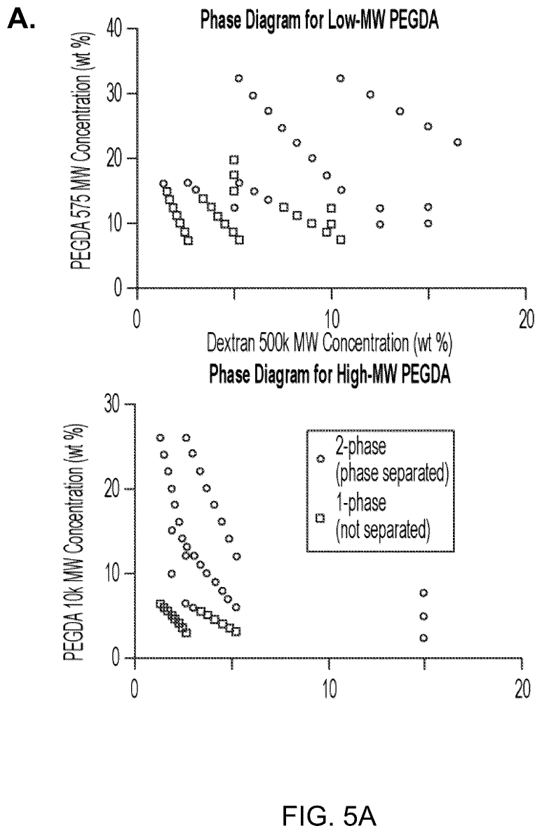

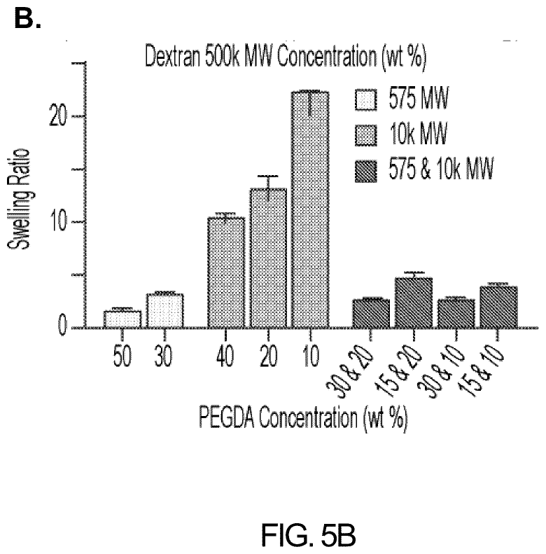

In order to inform selection of compositions of biocompatible materials that could enable ATPS-based phase separation, formation of stably biphasic structures, and an outer phase with sufficient cross-linking to minimize passive release of loaded drug, experiments were performed on materials in bulk. Specifically, compositions containing low and/or high MW (i.e. 575 Da and 10 k Da) PEGDA and high MW (i.e. 500 k Da) dextran that resulted in phase separation in bulk were examined (FIGS. 3A-3D). It was observed that high MW PEGDA (10 k Da), compared to low MW PEGDA (575 Da), induced phase separation starting at lower concentrations (FIG. 5A). Phases with the highest MWs and highest concentrations resulted in effective phase separation as shown by the phase diagram, and also separated quickly to enable phase separation prior to the photopolymerization region. It was also observed that in the presence of high MW dextran, a mixture of high and low MW PEGDA (“mixed MW” PEGDA) exhibited fast phase separation similar to high MW PEGDA.

Low MW PEGDA typically exhibits greater stiffness and faster photopolymerization, and high MW PEGDA typically has larger pore sizes. As an outer phase, a small pore size would be best suited to contain background release of payload in the inner phase. Testing a range of compositions of PEGDA, high MW PEGDA demonstrated a higher swelling ratio than that of low MW PEGDA, across a range of concentrations of PEGDA (FIG. 5B); using the measured swelling ratio, pore sizes for PEGDA were estimated at varied concentration and MWs, which confirmed low MW PEGDA having a smaller pore size than high MW PEGDA, as expected.

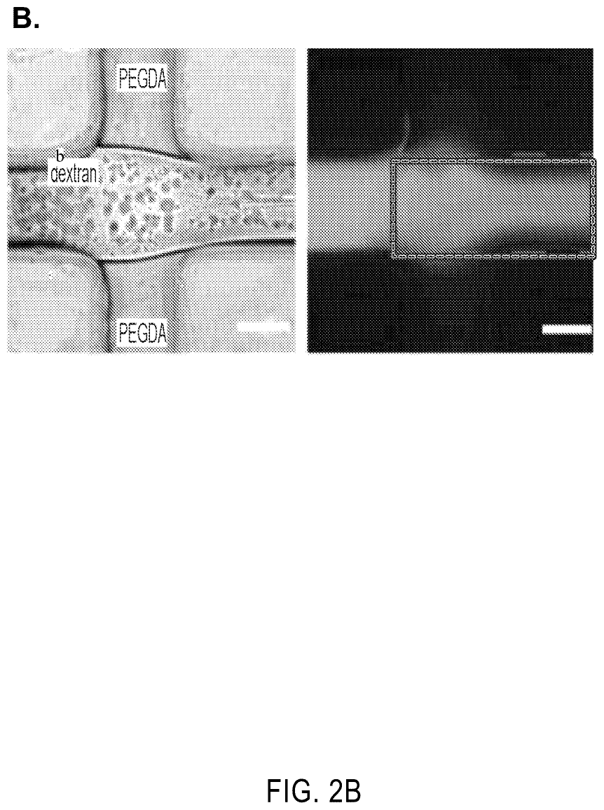

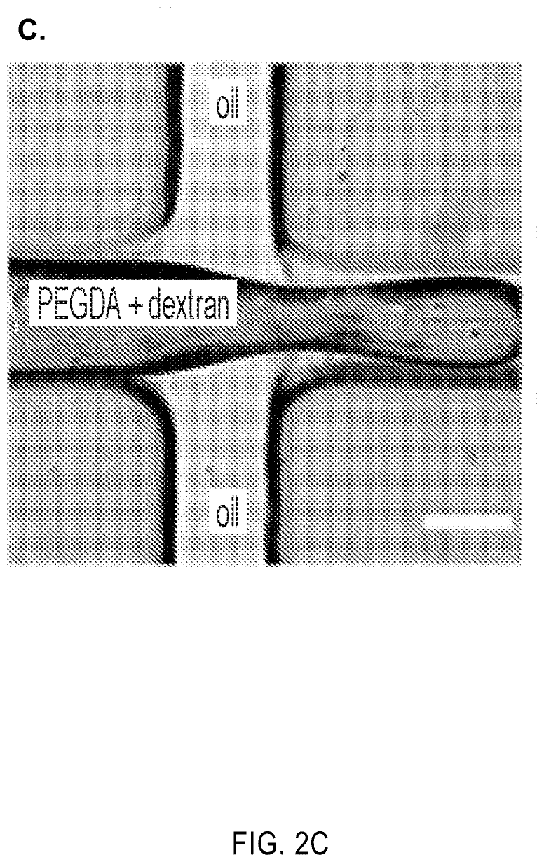

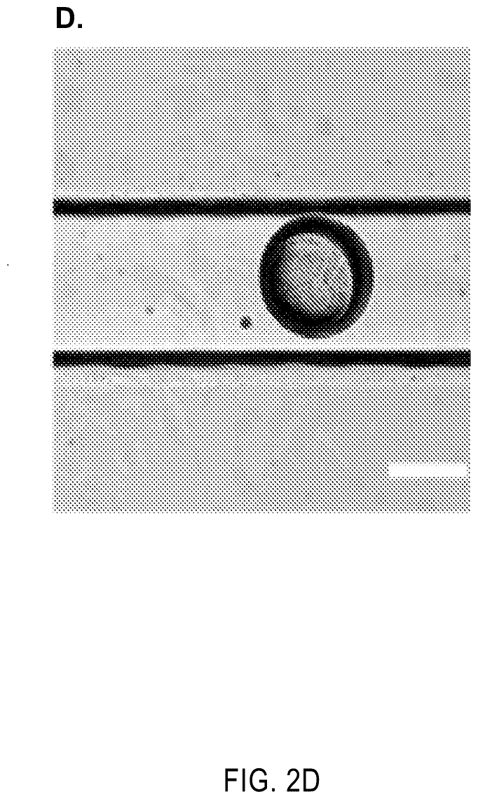

Next, PEGDA samples containing a mixture of high and low MW PEGDA were examined; these “mixed MW” PEGDA gels exhibited swelling ratios similar to that of low MW PEGDA (FIG. 5B). This result suggests that the mixed MW PEGDA, while exhibiting fast phase separation suitable for microfluidic fabrication, has a small pore size on the order of low MW PEGDA samples (consistent with a previous suggestion that low MW PEGDA fills into the voids of a composite MW sample). Additional PEGDA and dextran compositions were tested on the microfluidic chip, with different flow rates, and with different photoinitiators (FIG. 6). Amidst a range of compositions and relative amounts of inner and outer phase of microcapsule (as varied by changing gel compositions and flow rate), the overall asymmetric biphasic structure was consistently attained minutes after photopolymerization. However, 24 hours after fabrication, microcapsules made with low MW dextran did not retain their inner phases, while those made with high MW dextran retained their inner phases to produce a stably biphasic structure (FIG. 2B). Additionally, droplet formation at the interface between the aqueous phases and the oil regime was refined by tuning the concentration of SPAN™ 80, a surfactant (FIGS. 7B-7E).

### Example 2

### Swelling Ratio

To calculate the expected pore size of poly(ethylene glycol) diacrylate (PEGDA) at a range of molecular weights and concentrations, sample discs of PEGDA were prepared at both 10 kDa and 575 Da molecular weights, in a range of concentrations, with 10 mm diameter and 70 pL volume. 2.5% (w/v) Darocur 2959 was added to each sample. A custom PDMS well was used to contain samples while exposing the samples to UV light, at an intensity of 12 W/cm² for 5 seconds, with a 1″ spacer between the UV light source and the sample.

Once the samples were photopolymerized, samples were soaked in 1000 p L of deionized water in a 24-well plate for 24 hours, to wash off all unpolymerized monomers on the surface. Water was changed six times to facilitate removal of all unpolymerized material. After the washing period, wash water was removed from wells and prepared plates were placed in the vacuum desiccator overnight to allow for drying. Each sample was weighed to determine the dry weight, and then placed into fresh wells in a twenty-four well plate with 400 pL DI water. Each sample was subsequently removed from the surrounding solution as designated timepoints, blotted to remove excess water, and weighed. Each disc was then placed back into a well of DI water, and the weighing procedure was repeated twice a day for three days, until an equilibrium is confirmed, to observe swelling of the PEGDA structures over time.

### Example 3

### Phase Separation

To assess when a dextran-PEGDA mixture was in the two-phase regime or in the one phase regime (as plotted in FIG. 5A), dextran-PEGDA mixtures were prepared by vortexing and, and then allowing the samples to come to equilibrium for twenty-four hours. After twenty-four hours, each of the samples was assessed for a visibly-apparent boundary line between the phases (FIGS. 3A-3D).

### Example 4

### De-Mixing

To evaluate the temperature above which solutions of PEGDA and dextran phase separate, a dextran-PEGDA mixtures was prepared. 45 pL 50% PEGDA 575 Da was combined with 55 L 15% Dextran 500 k Da. The solutions were mixed together and allowed to phase separate overnight (FIG. 3C). The solution was then heated in a water bath to a temperature of 80° C. At 80° C., the solution was still below the critical de-mixing temperature and consisted of two phases (FIG. 3D).

### Example 5

### Pore Sizing

For two different molecular weight PEGDA solutions (10 k Da or 575 Da), the molecular weight between crosslinks were calculated for a variety of concentrations according to the following equation:

$$\frac{1}{{M}_{C}} = \frac{2}{{M}_{N}} - \frac{\frac{\frac{{ρ}_{s}}{{ρ}_{p}}}{{V}_{1}} ( ln ( 1 - {v}_{2} ) + {v}_{2} + {χ}_{1} {v}_{2}^{2} )}{{v}_{2}^{1 / 3} - \frac{{v}_{2}}{2}}$$

where MN is the average molecular weight of the un-crosslinked polymer, V/ is the molar volume of the solvent, V2 is the polymer volume fraction as calculated as the inverse of the volume swelling ratio, and A; is the polymer-solvent interaction parameter. The pore size was then calculated as:

$$ξ = {Q}_{v}^{1 / 3} {I ( {C}_{n} {N}_{b} )}^{1 / 2}$$

where Qᵥ is the volume swelling ratio, I is the length of carbon-carbon bond, Cₙ is the Flory characteristic ratio, and Nb is the number of carbon-carbon bonds between closest crosslinking points.

### Example 6

### Fabrication of Microfluidic Device

The molds for the PDMS portion of the microfluidic devices were fabricated on a Stratasys 3D printer; after printing, molds were washed to remove excess support material using a caustic soda-based cleaning solution, and then baked for at least 2 hours at 65° C. The PDMS prepolymer base (Sylgard 184, Dow Corning, USA) was combined with the curing agent at a weight ratio of 8:1. After hand mixing and 5 minutes of centrifuging at 3000 rpm, the PDMS was poured into the mold, degassed for 45 minutes, and then cured for 3 hours at 65° C. The cured PDMS was then removed from the mold and the inlet and outlet holes were punched with a 1.25 mm biopsy punch, and the chip was bonded via oxygen plasma treatment to a cleaned glass slide with a 100 pm layer of PDMS that had been deposited by spin coating. The bonded microfluidic chip was then heated at 90° C. for 1 hour.

The inlet channels were 500 microns in width and depth, flowing into a nozzle of 400 microns in width and depth, and then a serpentine channel with 650×650-micron dimensions. The microfluidic chip geometry was determined based on the end desired diameter of the microcapsules, with considerations for the appropriate geometry and flow rates for achieving co-jetting at the first junction and dripping at the second junction.

### Example 7

### Fabrication of Microcapsules

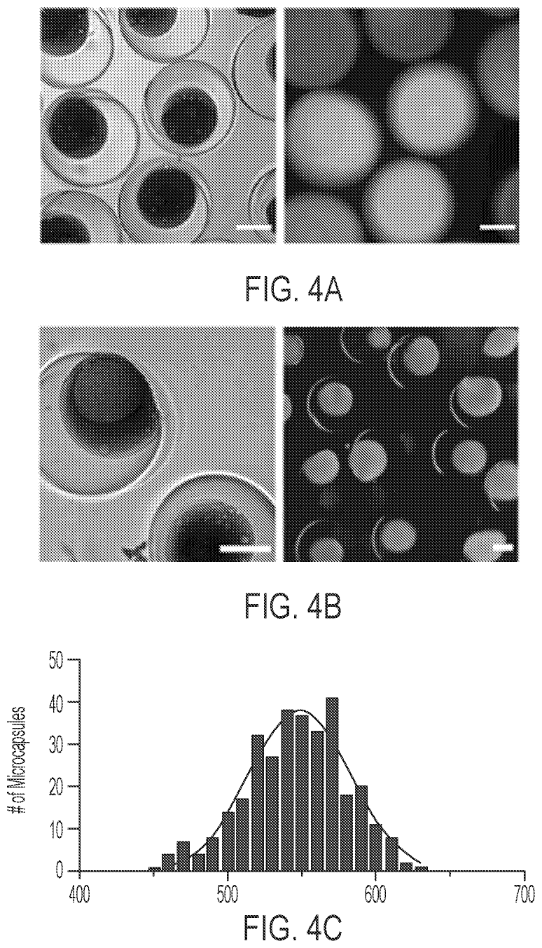

Double-emulsion microcapsules were fabricated using a microfluidic chip with two flow-focusing junctions in series followed by a region for photopolymerization. Flow rates were selected that resulted in the dispersed dextran solution jetting in the continuous PEGDA phase past the first junction (FIG. 2A). Immediately after the first junction, a small amount of mixing took place between the dextran and PEGDA phases, as shown in fluorescence imaging by a slightly diffuse phase boundary. At the second junction which meets an outer oil phase, the dextran-PEGDA co-flow broke into aqueous droplets (FIG. 2B). Discrete aqueous droplets flowed through the serpentine area of the microfluidic chip (FIG. 2C), during which phase separation occurred, until the region for photopolymerization. During the phase separation process, PEGDA and dextran are mixed together and subsequently phase separate, forming one phase that is primarily PEGDA and one phase that is primarily dextran. During this process, some amount of fluorescent compound enters the PEGDA phase. However, this fluorescence leaches out of the PEGDA phase after several days (as can be seen in the difference between FIG. 4A, taken shortly after fabrication, and FIG. 4B, taken 112 days after fabrication).

Next, the droplets underwent photopolymerization while in the serpentine channels of the microfluidic chip. During this process, the separated outer PEGDA phase photopolymerized under ultraviolet light at an intensity of 12 W/cm², forming an outer PEGDA structure that has low permeability to the model drug. These microcapsules have a stably biphasic structure, in contrast to monophasic crescent structures which use lower MW PEGDA and dextran. The inner phase was asymmetrically positioned with respect to the overall structure, which may be attributable to the large mismatch between the density of the inner dextran phase (500 k Da) and the outer PEGDA phase (575 and 10 k Da).

The dextran phase comprised 3.75% dextran 500 k with 2.5% FITC-Dextran 20 k as a model drug, and the PEGDA phase comprised 20% PEGDA 10 k and 30% PEGDA 575. The relative concentrations of PEGDA and dextran were tuned to allow for phase separation to occur on-chip. On-chip, the dextran channel had an inlet flow rate of 0.9 pL/min, the PEGDA channel had an inlet flow rate of 0.5 pL/min, and the oil phase had an inlet flow rate of 12 pL/min. The microcapsules were photopolymerized by applying UV light at 365 nm wavelength (OMNICURE® S2000 Spot UV Curing System, Excelitas Technologies).

### Example 8

### Ultrasound Configuration for FUS Generation

A function generator and transducer, with inset hydrophone, were used for the generation of focused ultrasound (respectively, TPO102, H102, and Y107, Sonic Concepts). The transducer had a 63.2 mm radius of curvature and was fitted with a 17.5 mm active diameter hydrophone in its central opening. A function generator (TPO102) was connected to a transducer (H102), with an impedance-matching network that matched the generator with the transducer's output impedance to maximize the electrical output power transmitted into the transducer.

The output from the hydrophone passed through a 5× preamplifier (SR445A, Stanford Research Systems) and then through a 1.8 MHz cutoff high pass filter (EF509, Thorlabs), with resulting data collected by an oscilloscope (SDS1202X-E, Siglent Technologies). The transducer was fitted with a polycarbonate coupling cone (C101, Sonic Concepts) filled with degassed water, and was placed within a degassed water bath for all experimental runs. All FUS applications were applied at 5% duty cycle and 150 W, unless stated otherwise.

### Example 9

### FUS Parameter Tuning

By adjusting ultrasound parameters, the dosage per FUS application could also be adjusted. The ultrasound parameters so adjusted consisted of application time (the total period during which FUS was applied), pulse length (length of each individual FUS pulse within an application time), duty cycle (percentage of time that ultrasound is on), applied intensity, and frequency (using either the fₒ frequency of 1.1 MHz or the fa frequency of 3.3 MHZ). The quantity of compound released could be increased by increasing power intensity (which results in higher acoustic pressure, larger peak negative pressure, and allows nuclei more time to grow via rectified diffusion) (FIG. 10D), or decreasing frequency (which reduces the period for change of direction in the pressure wave, and hence greater expansion and compression per pulse) (FIG. 10B). Generally, higher inertial cavitation occurs at lower frequencies, as cavitation nuclei can expand to greater radii due to the longer time for rarefaction. As such, cavitation nuclei in the microcapsule are more likely to undergo inertial cavitation at 1.1 MHz rather than 3.3 MHz, causing more payload release.

FUS can induce heating in a material. As an ultrasound wave passes through a material, the pressure wave causes localized shearing of the material, which can result in frictional heating. When the duty cycle of the applied ultrasound is high, there is insufficient time for the generated heat to dissipate, resulting in temperature rise. The acoustic power was chosen since the greatest amount of release was seen, all other parameters held constant. The low duty cycle (5%) allows time for generated heat to dissipate. This results in a high spatial-peak pulse average intensity, Isppa, which is the maximum intensity averaged over the pulse duration. A high Isppa is correlated with increased cavitation effects. Simultaneously, this results in a low spatial-peak temporal average intensity, Ispta, which results in low localized heating, which was seen in the low increase in temperature (FIG. 7F). When this set of FUS parameters were tested in a murine model, no adverse effects were detected.

Ultrasound is an attractive mechanism for triggering release, as it is commonly used in medical settings and is generally considered to be safe. Medical ultrasound often occurs at frequencies between 2 and 15 MHz, while this system primarily used a frequency of 1.1 MHz. The FDA limits Lptato 730 mW/cm² and Ispta to 240 W/cm². Off-target effects were expected to be minimal. The focal area of the transducer is small, with a cross sectional area less than 2 mm² and a depth of 10 mm; outside of this volume, the effects of FUS are reduced. Inertial cavitation is inversely proportional to frequency, and the relatively high frequency used (1.1 MHZ) would have minimized unintended cavitation.

### Example 10

### Signal Analysis

The time-domain signal detected by the hydrophone data was converted into the frequency-domain using Welch's method, which applies a moving window to take a discrete Fourier transform over segments of the signal and average the squared magnitude of the segments to determine a power spectral density (PSD) estimation (FIGS. 7B-7E)

### Example 11

### Imaging

A LEICA® DMI 6000B inverted microscope with 4× and 10× objectives, equipped with a motorized stage (Leica Microsystems, Bannockburn, IL) and LEICA® DFC9000 GT and DFC7000 T was used to acquire fluorescence and brightfield images. LEICA® LAS X software was used for image acquisition. Cropping, color adjustments and contrast enhancements of images as well as Z-stack projections were performed in ImageJ.

### Example 12

### Compound Release

During the compound release studies, effluent samples were collected before and after each FUS application, and transferred to a 384-well plate. The plate was analyzed for fluorescent intensity using a SYNERGY™ HI Hybrid Multi-Mode Reader (BioTek, Winooski, VT), at an excitation wavelength of 492 nm and an emission wavelength of 521 nm. The resulting data was expressed as arbitrary units (AU); Concentration was calculated using a standard curve, and then scaled by the number of microcapsules in each test well.

### Example 13

### Statistical Analyses

Statistical tests were performed in GraphPad Prism 9. Where data was assumed to be normally distributed, values were compared using a one-way ANOVA for single variable with a Sidak post-hoc test applied for multiple comparisons. Where data was assumed to be normally distributed, values were compared using a two-way ANOVA for more than one variable with a Tukey post-hoc test applied for multiple comparisons.

### Example 14

Fabrication and Triggered Release of Model Drug from Biphasic Microcapsules

Microcapsules were fabricated on a microfluidic chip using an aqueous two-phase system (ATPS), in which the two phases spontaneously separated above minimum polymer concentrations, when the decrease in enthalpy of de-mixing became greater than the gain in entropy of mixing. ATPS microcapsules were formed with biocompatible polymers and without intermediate oil-based phases, which can adversely affect biocompatibility.

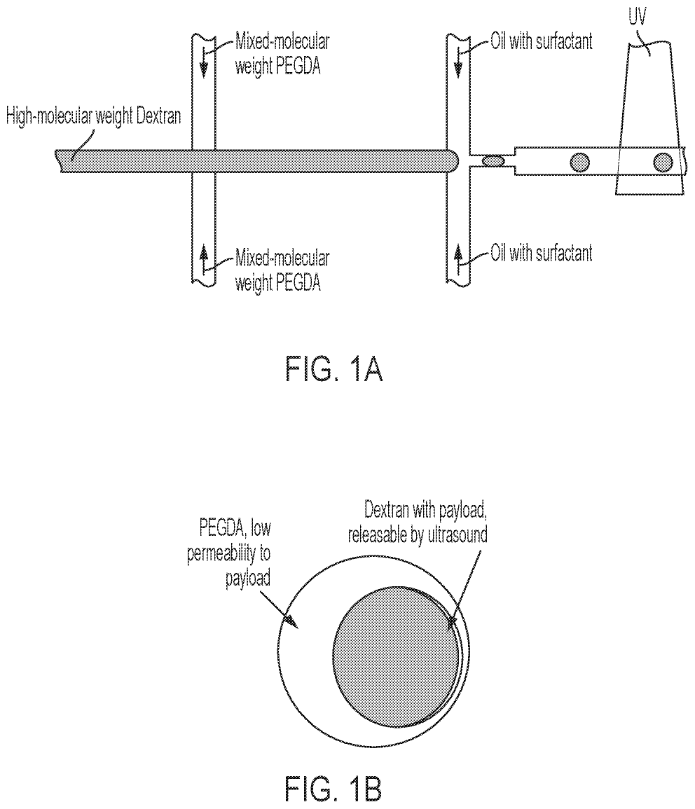

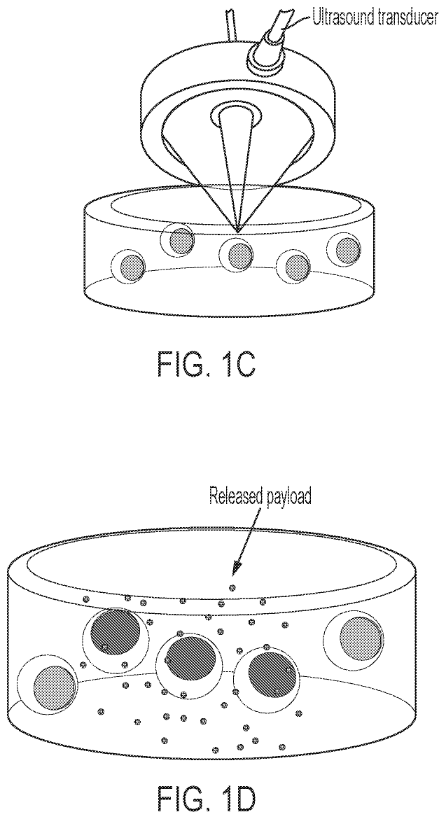

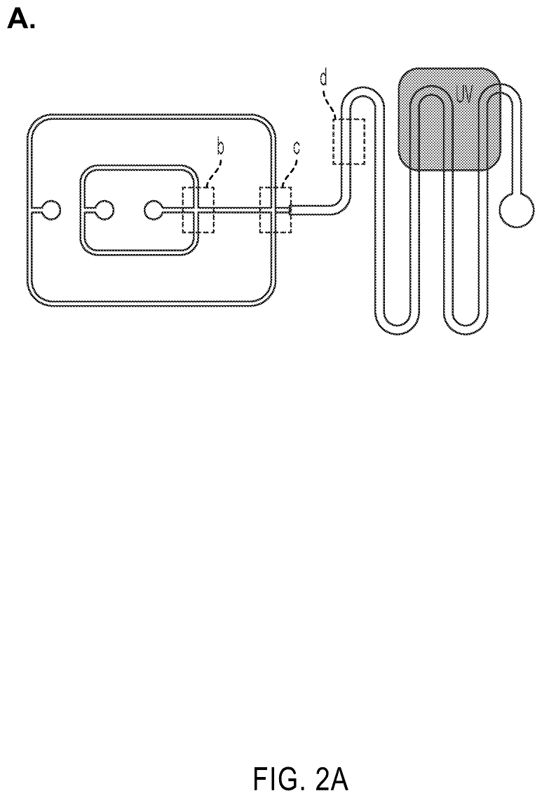

As shown in FIG. 1A, different molecular weights of PEGDA and dextran were used to form stable biphasic microcapsules. In this type of envisioned microcapsule (Figure IB), the outer phase (PEGDA) blocks passive release of payload stored in the inner dextran phase which remains intact until triggered by FUS. A focused ultrasound transducer, with a cross-sectional focal area smaller than 2 mm² and focal depth of 10 mm, selectively actuates the microcapsules (Figure IC). The inner phase of dextran remains intact until triggered by FUS, at which point it breaks down, releasing the payload (Figure ID). The application of FUS primarily causes breakdown of the soft inner dextran phase while also causing some breakdown of the stiff outer PEGDA phase. Compared to microbubbles (which are typically several microns in diameter), these biphasic microcapsules can carry large payloads and be used for pulsatile payload release over days.

Double-emulsion microcapsules were fabricated using a microfluidic chip with two flow-focusing junctions in series, followed by a region for photopolymerization (FIG. 2A). These polymerized microcapsules have a soft inner dextran phase and an outer PEGDA shell that has low permeability to the model drug, although there is a diffuse phase boundary. The effect of different materials on swelling and phase separation was tested in order to arrive at a final selection of materials (FIGS. 3A-3D). The microcapsules are biphasic with an asymmetric inner phase (FIG. 4A) with a minimally exposed surface to bulk solution. The microcapsules were morphologically stable after 112 days of storage in deionized water (FIG. 4B), with much of the fluorescent compound in the outer PEGDA phase having passively diffused out.

A set of fluid and flow conditions for delivery of payload were selected, and the consistency of the sizes of microcapsules generated was assessed. The innermost phase was high MW dextran loaded with fluorescein isothiocyanate (FITC)-dextran 20 kDa as a model drug, and the second phase was a mixed MW PEGDA. These coflowing phases were broken up into droplets by an outer oil phase. The flow rates were tuned so that the collected, washed microcapsules had an average diameter of 548.7±34.0 pm, similar to prior demonstrations on a percentage basis (FIG. 4C).

To assess ultrasound-responsiveness, microcapsules were transferred t into polydimethylsiloxane (PDMS) wells, with each well loaded with 24 microcapsules (corresponding to a cumulative delivery of ˜15 pg of compounds, suitable for clinical applications) in 25 pL of degassed deionized water. PDMS exhibits an acoustic impedance similar to that of water, minimizing the amount of wave reflected at the water/PDMS boundary and formation of standing waves. Wells were sealed with a thin plastic film and mounted onto the coupling cone of a 1.1 MHz focused ultrasound transducer (FIG. 7A). The transducer was powered by a variable drive box and data from the hydrophone was high pass filtered and amplified. An acoustic power of 150 W was used; this power, coupled with low duty cycle (5%), maximized cavitation effects while minimizing heating, and no adverse effects were detected when these parameters were used in a murine model.

Thermal effects from ultrasound application were monitored by placing a thermistor next to the microcapsule sample during select runs (FIGS. 7B-7E). At high duty cycle (20%), more than 4° C. of temperature rise was detected, compared to less than 2° C. at a low duty cycle (5%), with less than 1° C. of heating occurring during the first 10 s of the pulsed FUS at low duty cycle, indicating that minimizing the total application time further minimizes thermal effects (FIG. 7F). According to the model for calculating thermal dose, the effective thermal dose for all applied FUS conditions is below the threshold for cell death. For in vivo applications, the observed heating at low duty cycle in unlikely to damage tissue, particularly for short FUS applications (30 s or less).

Signal processing methods were used to analyze the cavitation occurring in and around microcapsules during FUS applications (FIG. 7G). In the frequency domain, spectral energy occurring at the harmonics of the fo frequency, 1.1 MHz, indicates emissions from stable cavitation. Additionally, the occurrence of spectral energy in the water-only sample is likely, in part, caused by the impedance change at the water/air interface on the top of the water bath. The spectral energy outside of those harmonic frequencies demonstrates broadband emissions, which were detected only when microcapsules were present (FIG. 7H). The detected broadband acoustic emissions during the application of FUS to microcapsules indicate inertial cavitation, as nucleation sites within the microcapsules collapse and cause shock waves, microjets, and micro streaming. These broadband noise emissions over the course of a single FUS pulse are apparent in spectrogram data, which exhibits increased intensities across a range of frequencies and over the duration of the pulse within the microcapsules sample, compared to the water only sample (FIG. 71). The spectrogram collected from microcapsules in the sample shows both greater harmonics and broadband emissions.

Finally, the effect of applied FUS on the structure and release of model drug from the microcapsule. As discussed previously, some amount of fluorescent dextran was mixed into the PEGDA phase before phase separation. Upon ten FUS applications, the dextran phase broke down (FIG. 8A), depending on the FUS intensity and duration of FUS application (FIGS. 8E-8F). Further applications of FUS, beyond when the dextran phase was disrupted, resulted in the PEGDA outer phase showing cracks and surface damage (FIG. 8B).

By applying 16 periods of 30 s pulsed FUS applications, 0.552±0.069 pg of a model drug was released from a microcapsule; without wishing to be bound by theory, the compound was presumably released through an area of inner phase exposed directly to solution. Over the same multi-day time period, a microcapsule passively released significantly less, 0.158±0.013 pg of model drug (FIG. 8C, left). After a period of equilibration in the first two hours in solution, during which most measured passive release occurred (FIGS. 9A-9B), FUS applications triggered the release of at least an additional 0.4 pg (FIG. 8C, right), which can take place over multiple doses and several days (FIG. 8D). The effect of FUS parameters, including power, frequency, and pulse length, on the amount of payload released and the overall stepwise profile were also studied (FIGS. 10A-10F).

While this invention has been disclosed with reference to particular embodiments, it is apparent that other embodiments and variations of the inventions disclosed herein can be devised by others skilled in the art without departing from the true spirit and scope thereof. The appended claims include all such embodiments and equivalent variations.

1. An aqueous biphasic microcapsule comprising:
a core comprising a payload-containing polymer; and
a shell encapsulating the core, wherein the shell comprises a photopolymerized polymer, wherein the payload-containing polymer and photopolymerized polymer are phase separated.

2. The aqueous biphasic microcapsule of claim 1, wherein the photopolymerized polymer comprises cross-linking which reduces passive release from the biphasic microcapsule of the payload.

3. The aqueous biphasic microcapsule of claim 1, wherein the photopolymerized polymer has a higher molecular weight than the polymer of the core.

4. The aqueous biphasic microcapsule of claim 1, wherein the drug-containing polymer and the photopolymerized polymer are biocompatible.

5. The aqueous biphasic microcapsule of claim 1, wherein the biphasic microcapsule comprises an asymmetric biphasic structure.

6. The aqueous biphasic microcapsule of claim 1, which comprises a diffuse phase boundary.

7. The aqueous biphasic microcapsule of claim 1, wherein said core is softer than said shell encapsulating the core.

8. The aqueous biphasic microcapsule of claim 1, wherein the core is asymmetrically disposed within the shell and the shell does not completely encapsulate the core.

9. The aqueous biphasic microcapsule of claim 1, wherein the payload-containing polymer core comprises a dextran.

10. The aqueous biphasic microcapsule of claim 9, wherein the core comprises an aqueous solution of about 3.75% to about 7.5% dextran having an average molecular weight of about 450 kD to about 650 kD.

11. The aqueous biphasic microcapsule of claim 1, wherein the photopolymerized polymer of the shell encapsulating the core comprises an aqueous solution of about 10% to about 20% of PEGDA having an average molecular weight of about 10 kD and about 5% to about 30% of PEGDA having an average molecular weight of 575 D.

12. The aqueous biphasic microcapsule of claim 1, wherein the photopolymerized polymer of the shell encapsulating the core comprises an aqueous solution of about 10% to about 20% of PEGDA having an average molecular weight of about 10 kD and about 5% to about 30% of PEGDA having an average molecular weight of 575 D.

13. The aqueous biphasic microcapsule of claim 1, wherein the payload comprises a drug or other therapeutic composition.

14. The aqueous biphasic microcapsule of claim 13, wherein the drug comprises an anti-cancer agent, a pain reliever, or insulin.

15. The aqueous biphasic microcapsule of claim 14, wherein the anti-cancer agent comprises a growth factor or cytokine.

16. (canceled)

17. The aqueous biphasic microcapsule of claim 1, wherein the interface between the core and the shell promote formation of nuclei suitable for inertial cavitation by ultrasound.

18. A plurality of aqueous biphasic microcapsules of claim 1, having an average diameter of from 450 mm to 630 mm.

19. A method of manufacturing an aqueous biphasic microcapsule, comprising:
(a) combining an aqueous solution comprising a first polymer, a second photopolymerizable polymer, and a photopolymerizing agent, in a microfluidic device so as to form a biphasic microcapsule comprising a core comprising the first polymer and a shell comprising the second photopolymerizable polymer, wherein the shell completely or incompletely encapsulates the core; and
(b) subjecting the biphasic microcapsule to a light source so as to photopolymerize the second photopolymerizable polymer,
thereby generating an aqueous biphasic microcapsule.

20. (canceled)

21. A method of delivering a drug to a subject comprising applying focused ultrasound (FUS) to a subject who has been administered one or more of the drug-containing aqueous biphasic microcapsules of claim 1, thereby triggering controlled release of the drug from the one or more biphasic microcapsules into the subject.

22. (canceled)

23. (canceled)

24. (canceled)

25. (canceled)

26. A method of seeding FUS-mediated cavitation in a location by applying focused ultrasound (FUS) to drug-containing aqueous biphasic microcapsules of claim 1 to the location.

27. (canceled)
